(19) United States
(12) Patent Application Publication
(10) Pub. No.: US 2025/0331755 A1
(43) Pub. Date: Oct. 30, 2025

(54) IMPLANTABLE AND FLEXIBLE CMOS RECORDING AND STIMULATING DEVICE WHICH INCLUDES ONE OR MORE NEURAL ELECTRODE ARRAYS

(71) Applicant: THE TRUSTEES OF COLUMBIA UNIVERSITY IN THE CITY OF NEW YORK, New York, NY (US)

(72) Inventors: Kenneth SHEPARD, New York, NY (US); Rizwan HUQ, New York, NY (US); Nanyu ZENG, New York, NY (US); Taesung JUNG, New York, NY (US); Brett YOUNGERMAN, New York, NY (US)

(73) Assignee: THE TRUSTEES OF COLUMBIA UNIVERSITY IN THE CITY OF NEW YORK, New York, NY (US)

(21) Appl. No.: 19/197,502

(22) Filed: May 2, 2025

Related U.S. Application Data
(63) Continuation of application No. PCT/US2023/036788, filed on Nov. 3, 2023.
(60) Provisional application No. 63/422,707, filed on Nov. 4, 2022.

(51) Int. Cl. A61B 5/268; A61B 5/00; A61B 5/279

(52) U.S. Cl. CPC A61B 5/268; A61B 5/0031; A61B 5/279; A61B 2560/0219; A61B 2562/164

(57) ABSTRACT

An exemplary system can be provide for facilitating electrophysiological recording and/or stimulation. The exemplary system can comprise a wireless neural interface device that can include a complementary metal-oxide-semiconductor (CMOS) integrated circuit. A flexible printed circuit board can also be provided with the system that can include a plurality of electrodes coupled to the CMOS integrated circuit. In addition, an exemplary method can be provided for manufacturing a wireless neural interface device for an electrical stimulation. According to such exemplary method, it is possible to provide a complementary metal-oxide-semiconductor (CMOS) integrated circuit that is mechanically flexible by being thinned. Further, it is possible to provide a flexible printed circuit board containing a plurality of electrodes. Then, it is possible to couple the flexible printed circuit board to the CMOS integrated circuit.

### CROSS REFERENCE TO RELATED APPLICATION(S)

This application relates to and claims the benefit of priority from International Patent Application No. PCT/US2023/036788 filed on Nov. 3, 2023 which was published as International Publication No. WO 2024/097404 on May 10, 2024, and from U.S. Provisional Patent Application No. 63/422,707, filed on Nov. 4, 2022, the entire disclosures of which is are incorporated herein by reference in their entireties.

### STATEMENT REGARDING FEDERALLY FUNDED RESEARCH

This invention was made with government support under Grant No N66001-17-C-4002, awarded by the Defense Advanced Research Projects Agency (DARPA). The government has certain rights in the invention.

### FIELD OF THE DISCLOSURE

The present disclosure relates generally to the technology of neural recording devices, and more particularly, to an implantable, preferably wireless, flexible CMOS surface recording device which can include one or more neural electrode arrays.

### BACKGROUND INFORMATION

Certain in vivo neural recording devices take the form of low-density electrode arrays that require wires to be run outside of the body, making these arrays cumbersome and prone to infection. The information acquired from these systems can be invaluable to the advancement of the understanding of the brain and in the development of neural prosthetic devices and brain-machine interfaces. However, in order to provide a better understanding of neuroscience and clinical neurology, there is a need for long-term implantable neural recording devices that offer many recording channels and a safe mode of data transmission.

One method for recording brain activity involves the use of electrophysiology. Electrodes permit both recording and stimulation, opening an avenue not just for understanding neural behavior but for actuating neural responses. Certain electrode arrays can require running wires through the skull and displace significant tissue volume, increasing inflammatory response. Scarring and gliosis is also increased by the rigid nature of certain electrodes and the rigid attachment of these electrodes to the skull.

In addition, microelectrode arrays can be fabricated using modern microelectronics processes, specifically complementary metal-oxide-semiconductor (CMOS) processes. Large numbers of surface recording and stimulating electrodes can be integrated with circuitry to condition recorded signals in an in vitro setting.

Implantable silicon-based neural interfaces leveraging CMOS technology have enabled bi-directional communication at unprecedented scales due to their ability to use of integrated circuitry (ICs) to multiplex among massive arrays of ultra-dense electrodes (see, e.g., References 1-3). However, their deployment in chronic clinical applications has often been limited by the inflammatory responses to the implants limiting signal integrity and percutaneous wires, which carry with them heightened risk of infection (see, e.g., References 4 and 5).

Thus, there is a need to address and/or improve such issues and/or deficiencies which exist in the previous devices, systems, and processes.

### SUMMARY OF EXEMPLARY EMBODIMENTS

Such issues and/or deficiencies can at least be partially addressed and/or overcome by providing fully implanted, wireless, flexible CMOS surface recording devices with neural electrode arrays according to exemplary embodiments according to the present disclosure.

According to an exemplary embodiment of the present disclosure, a system for electrical stimulation and recording is provided. The exemplary system can comprise a wireless neural interface device comprising a complementary metal-oxide-semiconductor (CMOS) integrated circuit, and a flexible printed circuit board containing a plurality of electrodes coupled to the CMOS integrated circuit. The CMOS integrated circuit can be thinned down such that it is mechanically flexible.

The exemplary wireless neural interface device can further comprise a radio transceiver for data transmission. In some embodiments, the radio transceiver can be contained on the CMOS integrated circuit. In other exemplary embodiments of the present disclosure, the radio transceiver can be contained on the flexible printed-circuit board.

The exemplary wireless neural interface device can also comprise circuits for wireless power transfer based on near-field inductive coupling. The circuits for wireless power transfer can be contained on the CMOS integrated circuit. The circuits for wireless power transfer can also be contained on the flexible printed-circuit board.

In some exemplary embodiments of the present disclosure, the system can further comprise an externally mounted relay station configured to wirelessly communicate with the wireless neural interface device. The exemplary system can also comprise an implanted relay station in the skull, spine or soft tissue under the skin, which is configured to wirelessly communicate with the wireless neural interface device. The exemplary system can also comprise an external computer connected either wirelessly or in a wire manner to the externally mounted or fully implanted relay station.

The plurality of electrodes can be or include an ultrathin polymer-based microelectrode array. The ultrathin polymer-based microelectrode array can be formed using thin-film microfabrication techniques. The plurality of electrodes can be or include a high resolution electrocorticography array for recording or stimulating at a neural surface. The plurality of electrodes can be or include penetrating electrodes and/or depth electrodes for recording and/or stimulating cortical layers or deep brain structures.

The exemplary CMOS integrated circuit can be thinned down using thinning techniques including backside grinding and reactive ion etching (RIE). The exemplary CMOS integrated circuit can be coupled to the flexible printed circuit board containing the plurality of electrodes through bonding. The bonding can be performed by forming solder bumps on bondpads of the CMOS integrated circuit, aligning to bondpads of the flexible printed circuit board, carrying out standard solder reflow techniques, and applying underfilling epoxy. The bonding can also be done by using anisotropic conductive film (ACF) or anisotropic conductive adhesive (ACA).

According to further exemplary embodiments of the present disclosure, devices and systems can be provided that can operate under a dual modality such as to be able to record and stimulate the surface of the brain and/or tissue in which they have been implanted. In addition, the density of the electrode arrays can exceed modern surface electrode arrays by several orders of magnitude, e.g., 2 or more. This density can be achieved without sacrificing the quality of recordings.

According to additional exemplary embodiments of the present disclosure, a method of manufacturing a wireless neural interface device for electrical stimulation and recording can be provided. The exemplary method can comprise providing a complementary metal-oxide-semiconductor (CMOS) integrated circuit that is thinned down such that it is mechanically flexible; fabricating a flexible printed circuit board containing a plurality of electrodes; and coupling the flexible printed circuit board to the CMOS integrated circuit.

The exemplary action of providing a complementary metal-oxide-semiconductor (CMOS) integrated circuit that is thinned down such that it is mechanically flexible can comprise thinning down the CMOS integrated circuit using thinning techniques including backside grinding and/or reactive ion etching (RIE). The exemplary action of fabricating a flexible printed circuit board containing a plurality of electrodes, can comprise forming the plurality of electrodes using thin-film microfabrication techniques.

The exemplary action of coupling the flexible printed circuit board to the CMOS integrated circuit, can comprise: forming solder bumps on bondpads of the CMOS integrated circuit; aligning the bondpads of the CMOS integrated circuit to bondpads of the flexible printed circuit board; carrying out standard solder reflow techniques; and applying underfilling epoxy. In other exemplary embodiments of the present disclosure, the action of coupling the flexible printed circuit board to the CMOS integrated circuit, can comprise bonding the flexible printed circuit board to the CMOS integrated circuit using an anisotropic conductive film (ACF) or an anisotropic conductive adhesive (ACA).

According to additional exemplary embodiments of the present disclosure, the method can further comprise encapsulating the CMOS integrated circuit using a chemical vapor deposition (CVD) process to deposit a conformal biocompatible exterior.

Indeed, according to various exemplary embodiments of the present disclosure, an exemplary fully-integrated flexible wireless neural interface platform can be provided that can be produce or otherwise be provided using exemplary wireless neural signal processing CMOS ICs incorporated into microfabricated polymer-based electrode arrays. Exemplary processes can also be provided—according to various exemplary embodiments of the present disclosure—for thinning a CMOS die to make it better suited for implantable application and for bonding this ultrathin die to a flexible electrode array.

For example, ultrathin die can be a significant improvement in interfacing with the biological world. Integrating these wireless die with flexible electrode arrays fabricated with thin-film techniques can improve chronic efficacy while prioritizing patient safety by eliminating dangerous percutaneous feedthroughs. Additionally, by provide an exemplary system for an exemplary design of the CMOS IC and the flexible electrode array, the exemplary technology can be applied to various potential applications, e.g., recording from the cortical surface to stimulating deep brain structures, applied to the peripheral nervous system, all with the basic process largely unchanged, etc., as well as for electrochemical sensing and targeted drug delivery to enhance understanding of the nervous system and facilitate treatment of a new generation of clinical diseases.

The exemplary embodiments of the present disclosure are not limited in any manner, and certainly can be utilized for brain interface(s), spinal cord (structure(s), as well as the entire central nervous system.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1C are sets of illustrations of various integrations of an ultrathin CMOS integrated chip (IC) with one or more flexible neural electrode arrays according to an exemplary embodiment of the present disclosure;

FIGS. 2A and 2B are illustrations of exemplary process flows for thinning CMOS ICs through bulk removal of the backside silicon in order to produce ultrathin chips (along with the images of the CMOS after various steps of the process) according to an exemplary embodiment of the present disclosure;

FIGS. 3A and 3B are illustrations of two respective process flows for bonding an ultrathin CMOS IC to a flexible electrode array according to an exemplary embodiment of the present disclosure;

FIG. 4A is a graph of measured somatosensory evoked potentials (SSEPs) recorded from a surface recording device in a porcine model according to an exemplary embodiment of the present disclosure;

FIG. 4B is an illustration providing an exemplary location of the device on the somatosensory cortex according to an exemplary embodiment of the present disclosure;

FIG. 4C is a set of exemplary graphs of the SSEPs;

FIG. 4D is an exemplary amplitude map with a lateral snout stimulation according to an exemplary embodiment of the present disclosure;

FIG. 5A is an exemplary trace (10 second) illustrating an exemplary cleanly isolatable neuronal firing from multiple neurons in the MUA band (e.g., 300 Hz to 5 kHz) in a multi-unit activity recorded on a surface recording device in a porcine model according to an exemplary embodiment of the present disclosure;

FIG. 5B is a graph of an exemplary instantaneous firing rate of the highlighted units (e.g., with Gaussian convolved) according to an exemplary embodiment of the present disclosure;

FIG. 5C is a set of illustrations providing mean and standard-deviation) waveforms (sub FIG. 5C(i) for all waveforms, (sub FIGS. 5C(i) and 5C(iii) for the highlighted units according to an exemplary embodiment of the present disclosure;

FIG. 6A is an exemplary chart illustrating an exemplary predictive capability of a classifier for SSEPs derived from snout stimulation according to an exemplary embodiment of the present disclosure;

FIG. 6B is a diagram and graph illustrating an improvement in the performance of the classifier with a bio-integration of the electrodes according to an exemplary embodiment of the present disclosure; and

FIG. 7 is a set of illustration of a device configured to perform a spinal cord stimulation and recording, in which the electrodes on a patient can be positioned epidurally dorsally on the spinal cord or wrapped around the ventral surface, according to an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the certain exemplary embodiments illustrated in the figures and the appended claims.

### DETAILED DESCRIPTION OF EXAMPLE EMBODIMENTS

The following description of exemplary embodiments provides non-limiting representative examples referencing numerals to particularly describe features and teachings of different aspects and exemplary embodiments of the present disclosure. The exemplary embodiments described herein should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the invention to such an extent that other implementations, not specifically covered but within the knowledge of a person of skill in the art having read the description of embodiments, would be understood to be consistent with an application of the exemplary embodiments of the present disclosure.

For example, the inflammatory response to neural implants can be greatly reduced by appropriately designing for the application (see, e.g., Reference 6).

First, this can be performed by minimizing or reducing the footprint of the implant by taking the functions once carried out by cumbersome rack-mounted laboratory equipment—such as signal amplification, filtering, and digitization—and custom-designing a single CMOS chip to perform most or all these roles in a vastly smaller form-factor. These CMOS dies can then be thinned from about 300 μm thick to 10 about μm, bringing implant volume to an absolute minimum without sacrificing utility.

Second, the mechanical mismatch (and resultant micromotion) between rigid silicon-based implants and the soft neural tissue has been implicated as an important driver of chronic inflammation leading to long-term signal degradation (see, e.g., reference 7). While aggressively thinning the CMOS die does confer some degree of flexibility, a far better mechanical match can be achieved by using a soft polymer-based electrode array as a bi-directional bridge between the CMOS chip and the neural tissue (see reference 8). Additionally, this modular design can facilitate a single custom CMOS chip design to function as the back-end for countless electrode arrays and applications.

Traditional wire leads used to connect neural implants to the external world in clinical applications, such as electrocorticography (ECOG), can also form direct avenues for dangerous infections. Fully implanted systems, such as e.g., deep brain stimulation (DBS) and responsive neurostimulation® (RNS®), can require battery powered implants and wires traversing the dura which pose risk of scarring, cerebrospinal fluid leak, and infection. In order to maximize patient safety, it can be important to provide purely wireless modalities to interact with neural interfaces. For example, advanced wireless power and data telemetry circuitry can be included directly on CMOS die, and used in conjunction with coils and antenna build into the chip itself or by incorporating these components onto the polymeric electrode array platform.

By addressing various limitations of state-of-the-art neural interfaces, flexible wireless CMOS-integrated electrode arrays according to exemplary embodiments of the present disclosure can facilitate the bi-directional interrogation of neural tissue while maximizing chronic viability and patient safety.

To provide such advantages, a neural implant with a wireless miniaturized integrated CMOS circuit set on an ultrathin soft polymer array according to the exemplary embodiments of the present disclosure can be provided, which can be a safe and durable wireless implant for neural recording and stimulation. Such exemplary arrangement can include a soft polymer-based electrode array and thinned down circuitry prevent signal degradation and inflammatory response. This exemplary arrangement can also contain devices for power and data telemetry making it wireless and more functional for long-term use. As such, with its flexible and minimal design, the exemplary embodiments of the present disclosure can make safe and long-term bi-directional neural communication possible in human patients.

According to various exemplary embodiments of the present disclosure, recording and stimulation can be provided and/or facilitated for the diagnosis and therapeutic management of many pathologies, which can include epilepsy, movement disorders such as Parkinson's disease and tremor, brain or spinal cord injury, cerebrovascular accidents, paralysis, hemiplegia, dementia and cognitive disorders, depression, anxiety disorders, post-traumatic stress disorders, loss of control eating, obesity, autonomic dysregulation. For example, brain-computer interfaces can be used for the management of disorder of moto function, vision, speech, and hearing, in accordance with the exemplary embodiments of the present disclosure.

As described herein, the exemplary miniaturized (CMOS) technology can include, but not limited to, e.g., a flexible array of sensing/stimulating electrodes that can facilitate neural signal recording and electrical stimulation. Such exemplary CMOS technology can combine, e.g., the flexible array with an ultrathin integrated circuitry die allowing for signal processing and wireless power and data use. The exemplary CMOS technology can also incorporate electrode post-processing techniques to enhance viability. Further, the exemplary embodiments of the CMOS technology can provide for an externally mounted relay station wirelessly connected to the implant, facilitate a brain-computer interface that is more biocompatible and minimally invasive, and/or used as a multi-functional neural interface. Further, the exemplary technology described herein can be used as a therapeutic tool for recording and electrical stimulation for: the central nervous system (epilepsy), the peripheral nervous system (motor dysfunction), surgery and physical therapy, and neural tissue engineering neurological disorders like epilepsy.

According to certain exemplary embodiments of the present disclosure, the implant device can comprise a flexible array of sensing/stimulating electrodes bonded to an ultrathin integrated circuit (IC) die capable of signal processing and wireless power/data. By integrating an ultrathin IC directly into the electrode array, the exemplary implant can leverages the exemplary miniaturized CMOS technology to incorporate sophisticated functionality in a biocompatible and minimally invasive form factor. By integrating radio frequency (RF) coils and antenna for power and data telemetry directly on the IC, the exemplary embodiments of the present disclosure can be used to receive programming commands, wireless power, and transmit wireless data without the use of bulky batteries or off-chip components. These wireless capabilities can be facilitated, e.g., by an externally mounted relay station which wirelessly couples to the IC and can be further connected to an external computer.

According to certain exemplary embodiments of the present disclosure, in addition to the flexible electrode array and IC, the exemplary embodiments of the present disclosure can incorporate electrode post-processing techniques to enhance sensing and stimulating neural tissue as well as biocompatible encapsulation to protect both the tissue and IC in order to reduce inflammation or circuit failure.

For example, according to the exemplary embodiments of the present disclosure, post-processing can be applied to process a commercially produced silicon IC into an ultrathin form factor in order to minimize the footprint of the implant, and render the rigid die flexible. By combining exemplary thinning techniques such as backside grinding and reactive ion etching (RIE), the silicon die can be thinned to less than 10 μm thick. Through the use of the exemplary thin-film microfabrication techniques, the exemplary polymer-based microelectrode array and biocompatible encapsulation layers can be made to be less than about 5 μm each, for a total implant thickness under about 20 μm.

According to further exemplary embodiments of the present disclosure, by adopting a modular approach, using the exemplary technology, it is possible significantly expand the use-case for a given neural signal processing IC. While in traditional monolithic silicon-based CMOS neural implants, the circuitry and electrodes may be inseparable, in a modular framework, arbitrary CMOS chips can be bonded to arbitrary electrode arrays. For example, according to certain exemplary embodiments of the present disclosure, a single CMOS IC capable of bi-directional signal processing and wireless power and data telemetry can be bonded to either a high resolution electrocorticography array for recording and/or stimulating at the neural surface, or be bonded to penetrating or depth electrodes for recording and/or stimulating, e.g., cortical layers or deep brain structures, all without lengthy and costly redesign of the CMOS IC.

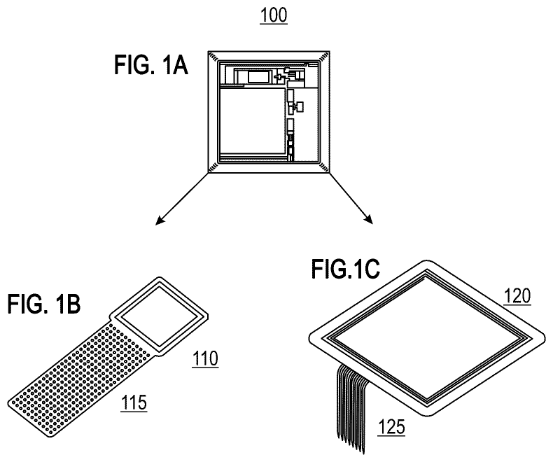

The exemplary embodiments of the present disclosure can facilitate the integration of ultrathin wireless-capable CMOS ICs with flexible neural microelectrode arrays. Exemplary implementations and modes of operation are shown in FIGS. 1A-1C, which provide illustrations of certain exemplary types of integrations of an ultrathin CMOS IC with flexible neural electrode arrays, according to exemplary embodiments of the present disclosure. An exemplary multi-functional neural interface IC with integrated wireless data and power capabilities is shown in FIG. 1A. In particular, FIG. 1A depicts the exemplary multifunctional neural interface IC 100 configured to record from and stimulating neural tissue while communicating with the external world through integrated wireless power and data capabilities. As shown in FIG. 1B, this exemplary IC is bonded to a flexible microelectrode array designed for electrocorticography, recording and/or stimulating tissue from the surface of the brain. FIG. 1C shows that the IC is bonded to a flexible array designed for penetration into the cortical layers of the brain. By designing the exemplary IC platform for a wide range of applications, many possible electrode array variants can be accommodated. For example, the exemplary flexible microelectrode array can take on any possible outer dimensions, as well as any electrode size and/or configuration to interface with a target area of neural surface of the brain, deep brain structures and/or spinal cord.

As described herein, the term multi-functional” can refer to, but not limited to, the ability to record from and stimulate neural tissue, perform signal processing tasks on the recorded data, digitize the neural signal, and finally interact with an external wireless relay station to receive power and exchange data. While the exemplary IC described herein can include or be associated with a power coil and data antenna fabricated directly on the chip as part of the CMOS foundry process, these exemplary elements can alternatively be included in the body of the flexible array instead, so long as the requisite circuitry is included in the exemplary CMOS IC.

The extensibility offered by the exemplary embodiments of the present disclosure significantly expands the use cases for appropriately designed neural recording ICs and flexible electrode arrays. An exemplary single CMOS IC design can function as the backend for any number of electrode configurations while in the conventional model, each variant would have to go through a costly months-long process of redesign, fabrication, and verification. By leveraging the exemplary IC and electrode array designs according to the exemplary embodiments of the present disclosure in this modular format, design iteration time and costs can be significantly reduced.

The illustration provided in FIGS. 1B and 1C show how an exemplary wireless neural IC 110, 120 can be used for both low frequency electrocorticographic surface recording and stimulation with millimeter-scale electrodes as well as high frequency action potential recording and stimulation with micron-scale penetrating electrodes. For example, FIG. 1B shows an non-penetrating example in which the exemplary IC 110 is bonded to a flexible microelectrode array 115 designed for recording and/or stimulating the brain cortical or spinal cord surface. The exemplary IC is bonded to a flexible biocompatible polymer-based ECoG array optimized for recording and stimulating large swaths of the cortical or spinal cord surface, ideally suited for scouting for and even disrupting irregular neural activity via closed-loop feedback. As shown in FIG. 1C, the exemplary IC 120 is bonded to a penetrating array 125 designed for interrogating small volumes of neural tissue deep within the cortical columns or even among subcortical structures, demonstrating capabilities that could bring about a wireless future for tens of thousands of DBS patients. By designing the exemplary IC platform for a wide range of applications, many possible electrode array variants can be accommodated.

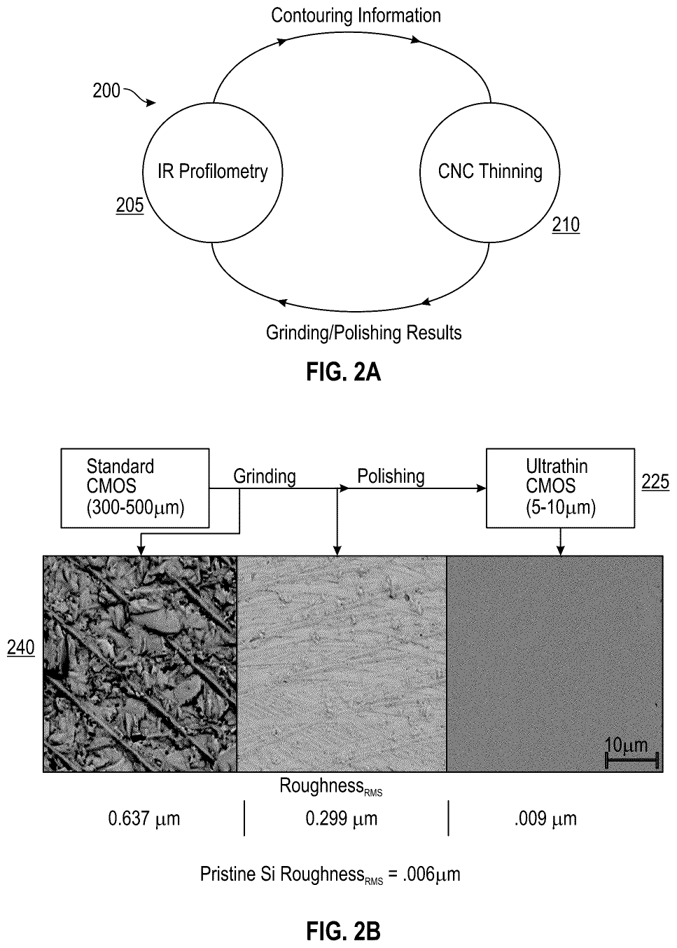

FIGS. 2A and 2B show illustrations of exemplary process flows for thinning CMOS ICs through bulk removal of the backside silicon in order to produce ultrathin chips according to exemplary embodiments of the present disclosure. In particular, FIG. 2A shows an iterative procedure 200 of using infrared (IR) profilometry 205 to collect contouring information which guides a computer numerical control (CNC) thinning procedure 210 which is then remeasured with IR profilometry 205 until the desired thickness is obtained. FIG. 2B shows exemplary surface roughness results 240 of one IC through the bulk thinning process, with a final surface roughness approaching that of a pristine CMOS IC provided from a commercial foundry.

Indeed, FIGS. 2A and 2B show exemplary approaches that can reduce the CMOS IC to a sub-10 μm thickness 225 in order to significantly decrease the footprint of the exemplary implant and maximize chronic viability. As shown in FIG. 2A, this can be done in an iterative process with IR profilometry and CNC-based thinning. For example, starting with a 300 μm thick die from a commercial foundry, contouring information can be collected to guide a coarse grinding operation to remove the bulk of the inactive backside silicon from the die. Once that grinding step is complete, the backside surface is reprofiled to guide a finer grinding step which removes far less material, but leaves a much smoother surface. This exemplary iteration can be repeated several times through finer and finer grinding and finally polishing stages, ultimately yielding a backside surface with roughness close to that of the original virgin silicon IC directly from the foundry as shown in FIG. 2B. However, such exemplary result may hide the presence of subsurface damage resulting from the grinding and polishing process, and an isotropic dry etch approach such as a reactive-ion etching (RIE) step may be employed at this point for additional stress relief to remove these high-stress damage sites.

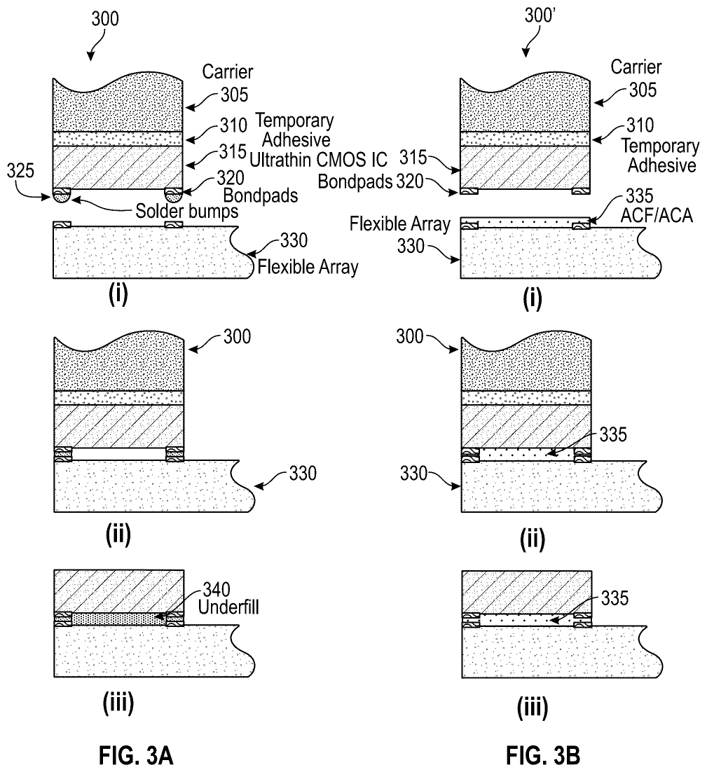

FIGS. 3A and 3B show illustrations of two respective process flows for bonding an ultrathin CMOS IC to a flexible electrode array according to exemplary embodiments of the present disclosure. As illustrated in FIG. 3A, this exemplary bonding can be performed by forming solder bumps 325 on the bondpads 320 of the IC 300, and attaching the thinned CMOS IC 315 to a carrier 310 using a temporary adhesive 310. In procedure (i), through the use of a flip-chip bonder, this assembly is then aligned to bondpads 320 of a flexible electrode array 330. Next, the bonding can be carried out through standard solder reflow techniques in procedure (ii). Further, an underfill epoxy 340 can be applied for a mechanical stability and the temporary adhesive 310 is released in procedure (iii).

As shown in FIG. 3B, for the IC 300′ shown therein, the bonding can be carried out in the absence of solder bumps through the use of anisotropic conductive film (ACF) or anisotropic conductive adhesive (ACA). Alignment and thermocompression bonding with the ACF/ACA can be performed with a flip-chip bonder. Indeed an unbumped CMOS IC 315 can be bonded to a flexible electrode array 330 using an anisotropic conductive film or adhesive, forming vertical electrical connections between raised bondpads without lateral shorts between adjacent pads. This can be done in FIG. 3B by applying the ACF/ACA to the substrate and aligning the components using a flip-chip bonder in procedure (i) and bringing the components in contact for thermocompression bonding in procedure (ii). What follows is a release of the temporary adhesive 310 and/or the carrier in procedure (iii) of FIG. 3B.

Following the bonding procedure, the implantable device can further be encapsulated to protect both the electronics and the biological environment. This can be done by a chemical vapor deposition (CVD) process to deposit a conformal biocompatible exterior that can be composed of an organic layer, an inorganic layer, or a multi-layer stack combining the barrier properties of multiple materials. Further, the encapsulant can be selectively removed from the electrode sites, and depending on the application of the electrodes, additional materials can be deposited onto the electrodes through spin-coating or electrodeposition processes to shape their electrical and mechanical properties.

The exemplary embodiments of the present disclosure can provide an exemplary system that can include a fully implanted, wireless, flexible CMOS surface recording device and a relay station. Specifically, in some embodiments, the device can include a flexible electrode array that is implanted in the region of interest (e.g., brain), in order to detect the desired signals. For example, the device can be implanted in the visual cortex to significantly improve quality of life for people suffering from blindness. For example, electrical stimulation of the human visual cortex using the exemplary device can yield the perception of small spots of light, known as phosphenes. In some exemplary embodiments of the present disclosure, the small feature sizes and massive scale of device can provide the opportunity for patients to perceive and discriminate complex patterns at higher resolutions.

According to further exemplary embodiments of the present disclosure, the exemplary device can include a band-pass filter that provides antialiasing for the subsequent digitization of the recorded signals as well as initial noise reduction from potential recording noise resulting from the flexible electrode array. The exemplary device can include one or more amplifiers to increase the power/amplitude of the recorded signals prior to performing analog-to-digital conversion using an analog-to-digital converter (ADC).

For example, upon recording and conditioning the analog signal, the recordings from the flexible electrodes are digitized through the use of low-power analog-to-digital converters (ADC). In some exemplary embodiments of the present disclosure, a dedicated ultra-low power ADC can be provided for each channel in the block currently being addressed. For example, time-division multiplexing in conjunction with an ADC that samples at a much faster rate that allows multiple channels to share a single ADC can be provided. This can facilitate fewer ADCs at the expense of power. According to further exemplary embodiments of the present disclosure, a successive-approximation register (SAR) or pipeline ADC architecture can be used. These exemplary architectures are feasible since relatively low sampling rates are required for individual channels.

Furthermore, in some exemplary embodiments of the present disclosure, the exemplary device can include a wireless power circuit to ensure that the implanted device and relay station are powered. According to still further exemplary embodiments of the present disclosure, the exemplary device can be powered via batteries, capacitors, energy harvesting circuits or any other suitable combination thereof. The exemplary device can include additional control logic devices for controlling operations of the device. For example, the exemplary device can include a stimulation control logic unit for controlling the flexible electrode array when it is operating in a stimulation mode. The exemplary stimulation control logic unit can provide commands to one or more blocks of the flexible electrode array associated with a stimulation pattern in a region of interest. In addition, the device can include a digital control logic unit that generates control commands for the overall operation of the device. For example, the exemplary digital control logic unit can determine the switching of the operation of the flexible electrode array from a sensing/recording operation mode to a stimulation operation mode. In some exemplary embodiments of the present disclosure, the exemplary digital control logic unit can automatically detect the appropriate switching times and associated parameters for the flexible electrode array. In some embodiments, switching can be performed manually or in any other suitable manner.

Furthermore, the relay station of the exemplary system can wirelessly exchange data and power with the device. In some exemplary embodiments of the present disclosure, the relay station can be or include a transceiver that can be situated outside the body positioned against the head in a cap or in any other suitable wearable manner and has a small form factor. In some exemplary embodiments of the present disclosure, the relay station can be encased and implanted in the skull, spine, or soft tissue under the skin. In some exemplary embodiments, the relay station can transmit data from the device to a base-station (not shown) for an off-chip analysis. The exemplary base-station can be or include a computer, a smart phone, a server and/or any suitable hardware processor.

Further, the exemplary external relay station can operate at a high-power budget when located outside the body and can be easily heat-sunk. For example, in some embodiments, the relay station can incorporate an 802.11n chipset and a 3.7-V, 4.2-Amp-hour lithium-ion battery thus consuming approximately 15 W when active and about 50 mW in standby, giving about 1 hour of activity on a battery charge. In addition, with the above parameters, the total weight for the relay station (e.g., wireless transceiver) can be about, e.g., 5 g, dominated by the battery and the total board size can be approximately 100 cm². In some exemplary embodiments of the present disclosure, the link between the relay station and other 802.11n wireless devices can be secured using the WPA2 standard or any other suitable encryption standard. In addition, in some embodiments, the link between the relay station and the device can be secured with secret keys, but the very short-range nature of this link can make this unnecessary.

In addition, in some exemplary embodiments of the present disclosure, the wireless power circuit can be or include a wireless RF link operating in near-field at about 13.56 MHz, delivering ˜300 mW to power both its stimulation/recording circuitry and two far-field impulse-radio ultra-wide-band (IR-UWB) microwave data links operating at a rate of 100 Mb/s. In some exemplary embodiments of the present disclosure, the data downlink from the relay station to the device shares the same antenna as the uplink with an on-off-keying modulation approach achieving a data rate of 50 Mb/s. The exemplary external transceiver can be battery powered, and can communicate to the outside world using, for example, the 802.11n protocol. The exemplary flexible microelectrode array can employ non-penetrating, high-density electrodes that can perform in a dual operation mode.

For example, in some exemplary embodiments of the present disclosure, the flexible microelectrode array can provide capacitive sending and stimulation of the desired region, which allows full dielectric encapsulation of electrodes for long-term stability. The exemplary use of conducting polymers or high surface area coatings can yield capacitances as high as 60 fF/μm² for the flexible microelectrode array. In addition, the exemplary fabricated flexible microelectrode array can provide non-penetrating electrode arrays to channel counts more than three orders of magnitude higher than the current state-of-the-art (e.g., to realize channel/electrode density exceeding 2500 electrodes/mm²), through use of active CMOS arrays.

Moreover, the exemplary relay station can provide power to the CMOS chip and relay data both to and from the implanted device. Specifically, in some exemplary embodiments of the present disclosure, in order to allow for the device to be fully implantable, antennas can be fully integrated onto the flexible CMOS chip and/or the flexible microelectrode array. For example, one set of antennas can operate to receive power at a lower carrier frequency (e.g., approximately 13.56 MHZ) and another set of antennas can be used to transmit and receive digital information using an ultra-wide band link at a center frequency of, e.g., approximately 4 GHz. In some exemplary embodiments of the present disclosure, the sets of antennas can operate two decades apart as to reduce interference and maintain fidelity of the transmitted signals. In addition, such exemplary configuration can facilitate for no battery to be incorporated into the implanted device.

Furthermore, due to certain design restrictions associated with the region of implantation, it can be preferable for power dissipation by an implanted device in the brain to be kept below, e.g., 500 μW/mm2, to insure local tissue heating below about 1° C.. In some exemplary embodiments of the present disclosure, in order to isolate power and data transfer signals, a multi-antenna solution can be employed. For example, the exemplary antennas can be completely integrated onto the CMOS die. These exemplary power and data links operate over two decades apart in frequency to avoid interference. For example, for power transfer, the ISM band at about 13.56 MHz with a coil that completely surrounds the outer circumference of the integrated circuit can be used. In some embodiments, a series resistance in the receiving coils can be reduced by the use of centimeter-scale, wide metal to limit losses.

In some exemplary embodiments of the present disclosure, for high rate data transfer off the chip, ultra-wide-band (UWB) techniques can be used. For example, impulse radio (IR) UWB can provide a simple, digital-circuit-style implementation to provide data rates as high as 500 Mb/s. In some exemplary embodiments of the present disclosure, an exemplary IR-UWB system can be centered at 4 GHz with a 900-MHz bandwidth (e.g., approximately 3.1 GHz to 4.9 GHZ). For example, this bandwidth can facilitate the use of 1.1 ns transmission pulses with 10 ns of reset time before the next pulse. In some exemplary embodiments of the present disclosure, a wideband, differential dipole antenna can be used to transmit the data to the relay station. For example, a ‘1’ can be encoded using a 1-ns burst of a 4 GHz square wave, and a ‘0’ can be encoded with silence. In some exemplary embodiments of the present disclosure, bursts can be sent, e.g., every 10 ns, corresponding to a data rate of about 100 Mb/s. Further, differential edge combining can be used to generate the UWB signal. For example, a 125 ps pulse can be generated on the rising edge of a ‘1’ using an edge detector circuit and a series of matched delay elements can provide 8 edges, spaced about 125 ps apart. In such exemplary cases, the delayed edges can be combined with a series of differential drivers to provide the current to the antennas.

According to certain exemplary embodiments of the present disclosure, the incoming data from the CMOS device can be received at the relay station with a matched antenna. For example, the received UWB modulated data can be filtered and rectified to extract the envelope. In some embodiments, the filtered signal can be buffered with a low noise amplifier and fed into a comparator. Since the exemplary modulation scheme is impulse radio based, the comparator converts the signal into the digital domain. The digital signal pulses can then be elongated before driving a clock and data recovery circuit. As discussed herein, the exemplary thinned, flexible CMOS devices can be provided that can become pliable when made sufficiently thin. A variety of thinning methods can be employed to support a three-dimensional (3D) integration of thin silicon stacks. For example, chemical-mechanical polishing (CMP), wet etching, and dry chemical etching (DCE) can be utilized to produce a CMOS chip less than 10 μm in total thickness, giving them the necessary pliability associated with the prescribed design requirements.

The exemplary flexible microelectrode array can be or include a passive electrode array fabricated using conducting polymer-based, poly(3,4-ethylenedioxythiophene) doped with poly(styrenesulfonate) doped with poly(styrenesulfonate) (PEDOT:PSS) integrated in a 4-μm-thick parylene film. In addition, PEDOT:PSS as electrode material offers ionic and electronic conductivity (e.g., 20 μm×20 μm recording sites exhibit 30Ω impedance at 1 KHz, as well as biocompatibility and chemical stability). In some exemplary embodiments of the present disclosure, iridium oxide can be used for fabricating the flexible microelectrode array or any other suitable electrode material.

Furthermore, a biocompatible, FDA approved class-6 polymer such as Parylene can be used for the passivation of the chip. Parylene is a chemically inert material and can provide electrical insulator. Upon thinning, the flexible microelectrode arrays possess adequate mechanical strength to be readily manipulated by the experimenter yet facilitate a high conformability permitting neuron-size electrodes to attain excellent electrical and mechanical contact with the curvilinear surface of the brain or any other suitable tissue. In some exemplary embodiments of the present disclosure, the flexible microelectrode arrays can be patterned from polyimide or dissolvable silk substrates, thus relying on external measurement electronics.

For example, in order to make the device implantable for long-term use, the device is fabricated to displace very little volume. Specifically, the thinned chip is highly pliable and provides high degree of optical transparency (>60%), which allows for the simultaneous optical and electrical recording. As discussed above, in some embodiments, to achieve the desired design requirements the CMOS device is thinned using mechanical and chemical techniques to, for example, a total thickness of less than 15 μm providing an integrated circuit with a significantly increased mechanical compliance that can conform to the surface of the brain. In some embodiments, after thinning, a passivation layer consisting of aluminum-oxide-parylene multilayers is deposited to seal the device and make it biocompatible for implantation.

Furthermore, the exemplary CMOS device can be rendered highly-flexible by extreme thinning so that the surface recording array is in conformation to the desired tissue e.g., pial surface of the brain. Specifically, bending stiffness for these dice, can scale roughly with the cube of the film thickness, allowing a reduction in die bending stiffness from roughly 1.76 Nm for a standard 500 μm thick die to only 5 μNm for a fully thinned die. As a result, the six-order-of-magnitude increase in mechanical compliance can facilitate the device to tightly conform to the curvilinear surface of the brain. In addition, in some embodiments, one UWB antenna can be used for the data uplink to improve the data rate without excessively increasing complexity. Further, both the transmit (TX) and receive (RX) RF transceiver designs can use a differential dipole antenna for both RX and TX of data. In some embodiments, each antenna can be tuned to 4 GHz and can be designed to transmit 100 Mb/s of data. For example, due to the smaller reticle size, the TX/RX Antenna of the device can have a hook shape but can still effectively act like a differential dipole antenna.

According to further certain exemplary embodiments of the present disclosure, the exemplary system can include a base-station for performing off-chip signal analysis of the raw signals recorded by the flexible microelectrode array. For example, in sensory applications, data analysis can consist of two important transforms: the signal transform, which maps neuronal activity to electrical signals recorded at the array (and, by reciprocity, permits defining spatiotemporal stimulation patterns from the massive array to target specific neurons) and the percept transform, which maps neuronal activity in subcortical layers to experiences that are both perceived and induced in the subject. In some embodiments, the signal transform can either be implemented fully in software or partially on chip. In addition, such signal processing can be at least partially or fully automatic (requiring no user intervention) and real-time, as it will not be possible to store the data offline for subsequent analysis.

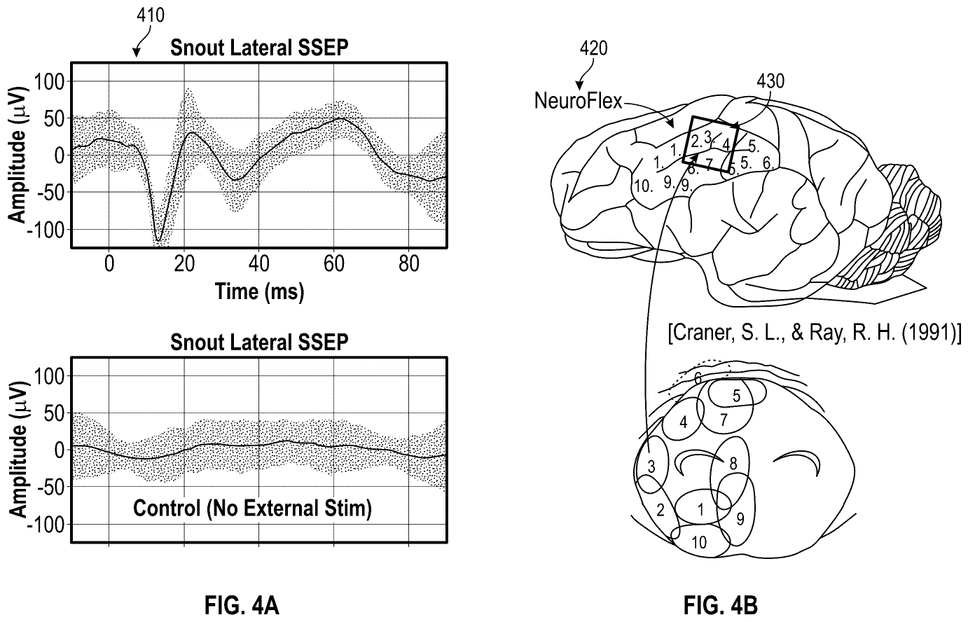

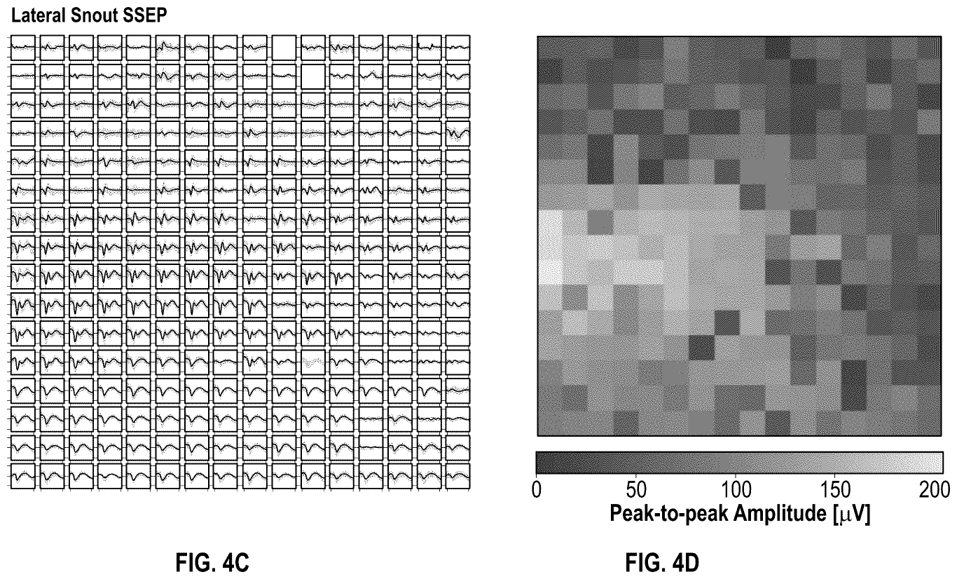

For one exemplary device positioned of the somatosensory cortex according to an exemplary embodiment of the present disclosure, the snout of a pig can be stimulated at various locations using but not limited to, for example, a clinical pulse generator (e.g., XLTEK Protektor32, Natus Neuro, Middleton, WI, USA). As shown in FIG. 4(a), the resulting exemplary somatosensory evoked potentials (SSEPs) 410 can be recorded, e.g., with a NeuroFlex device 420 positioned over a somatosensory cortex 430 as shown in FIG. 4B. FIG. 4C shows exemplary traces recorded over the entire 1024-channel array with an associated SSEP amplitude “heat map” that is illustrated in FIG. 4D.

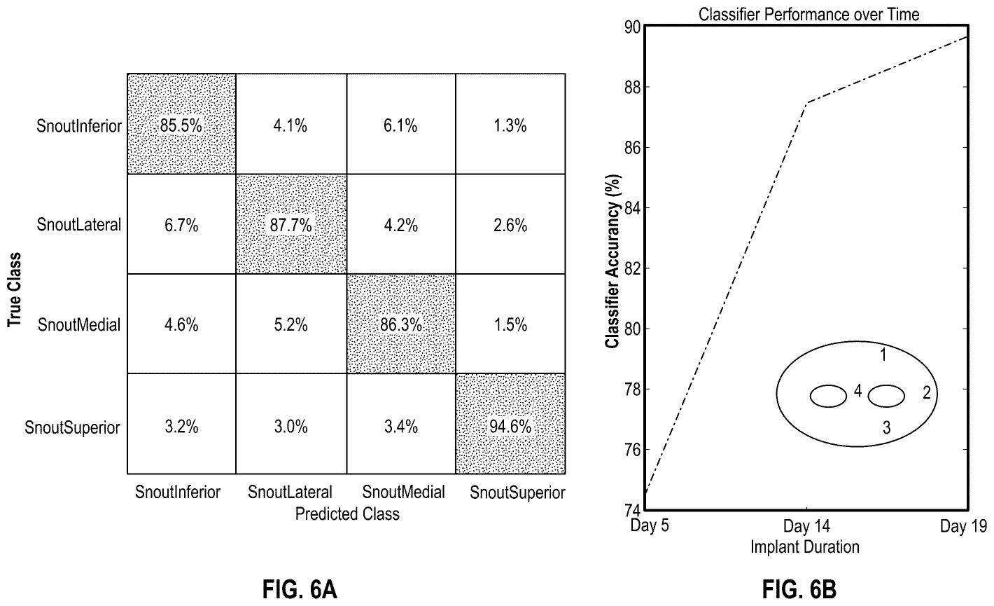

FIG. 6A shows a table providing an exemplary classifier based on a high-resolution somatotopic mapping of recorded SSEPs that was highly predictive of the corresponding region of snout electrical stimulation, according to an exemplary embodiment of the present disclosure. For example, heat maps (an example of which is shown in FIG. 4D) were indicated to be visually distinct and provided stable somatotopic patterns for each region of stimulation. As shown in FIG. 6B which illustrates a diagram and a graph indicating an improvement in the performance of the classifier with a bio-integration of the electrodes according to an exemplary embodiment of the present disclosure. It was also confirmed that classifier accuracy improves with the number of active channels. This can mean that the somatotopic recording of SSEPs improves with greater bio-integration, a phenomenon commonly observed with implanted electrodes. No signal degradation was observed.

In some certain exemplary embodiments of the present disclosure, after filtering raw signals using the bandpass filter from the recording microelectrode array to eliminate out-of-band information, a time-frequency dictionary-learning method can be used in order to compress non-action-potential (AP) signals for low-bandwidth transmission off-chip. Additional AP features can be identified during such process. For example, online scalable stochastic Dirichlet process variational inference methods or any other suitable method can be used to perform clustering on highly-dimensionally-reduced representations of the spiking event waveforms.

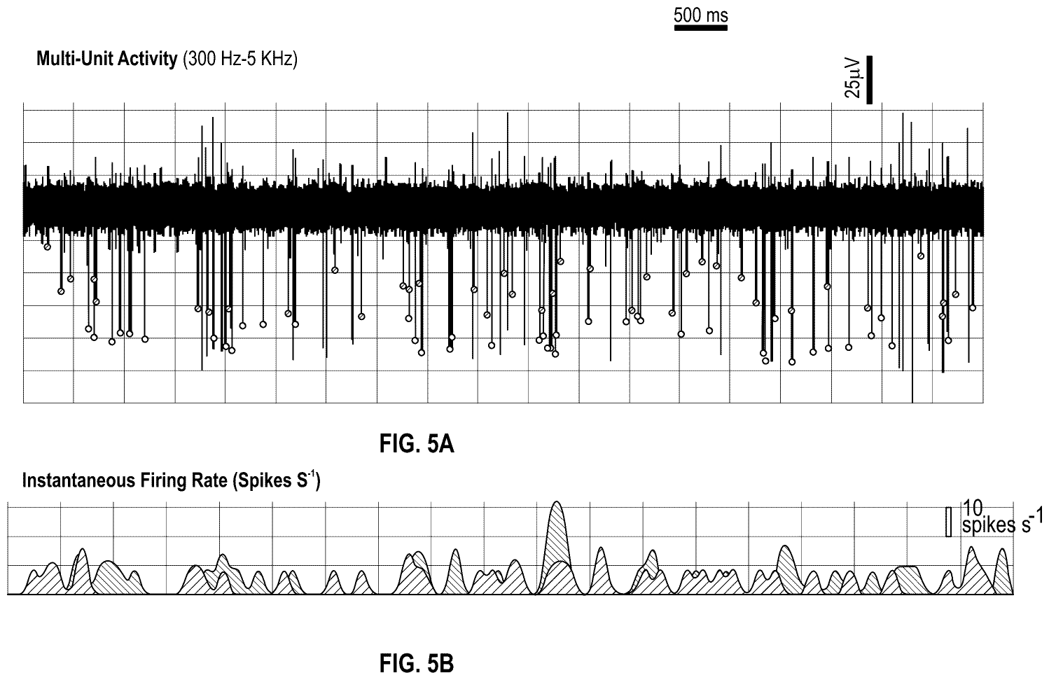

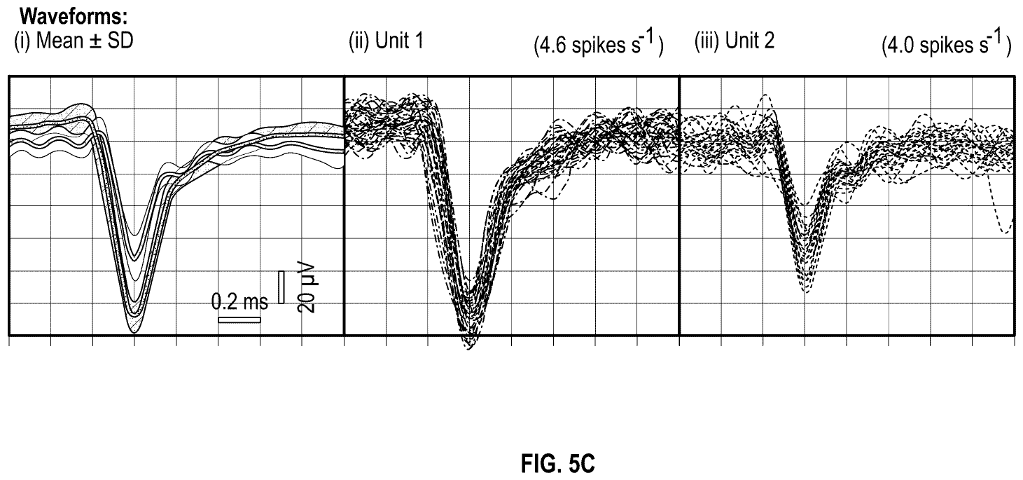

Multi-neuronal “collision” events can be identified, and subsequently eliminated using efficient orthogonal matching pursuit methods. In addition, Kalman tracking of the mean waveform can robustly and efficiently handle data non-stationarity. According to various exemplary embodiments of the present disclosure, the output of such analysis can provide spike times and identities, along with Bayesian measures of confidence about the timing and identity of each detected event. Furthermore, “spikes” here will be broadly defined, since in many cases these will be weaker signals than conventionally associated with AP features. In addition, the time-varying cluster means or subsampled detected spike waveforms can also be obtained, for offline model checking and validation. FIGS. 5A5C show exemplary graphs with exemplary representative spikes from multi-unit activity measured from an exemplary device of the present disclosure. In particular, FIG. 5A illustrates an exemplary trace (10 second) illustrating an exemplary cleanly isolatable neuronal firing from multiple neurons in the MUA band (e.g., 300 Hz to 5 kHz) in a multi-unit activity recorded on a surface recording device in a porcine model according to an exemplary embodiment of the present disclosure. FIG. 5B provides a graph of an exemplary instantaneous firing rate of the highlighted units (e.g., with Gaussian convolved) according to an exemplary embodiment of the present disclosure. Further, FIG. 5C shows a set of illustrations providing mean and standard-deviation) waveforms (sub FIG. 5C(i) for all waveforms, (sub FIGS. 5C(i) and 5C(iii) for the highlighted units according to an exemplary embodiment of the present disclosure.

According to additional exemplary embodiments of the present disclosure, the computer station can be equipped with on-line software capable of learning patterns in the recorded neural data in real time. Clustering of high dimensional data can be achieved by modern Dirichlet process variational inference methods. For example, by extracting features and identifying action potentials, the off-chip system can accurately correlate and investigate neural activity. An exemplary control feedback loop can be provided such that in response to certain events, the system can be programmed to generate appropriate recording and or stimulus patterns for experiments as it observes certain motifs in the signals.

According to exemplary embodiments of the present disclosure, a system for electrical stimulation and recording is provided. The exemplary system can comprise a wireless neural interface device comprising a complementary metal-oxide-semiconductor (CMOS) integrated circuit, and a flexible printed circuit board containing a plurality of electrodes coupled to the CMOS integrated circuit. The exemplary CMOS integrated circuit can be thinned down such that it is mechanically flexible.

The exemplary wireless neural interface device can further comprise a radio transceiver for data transmission. In some exemplary embodiments, the radio transceiver can be contained or otherwise provided on the CMOS integrated circuit. The exemplary radio transceiver can be contained on the flexible printed-circuit board.

According to further exemplary embodiments of the present disclosure, the exemplary wireless neural interface device can also comprise circuits for wireless power transfer based on near-field inductive coupling. The circuits for wireless power transfer can be contained on the CMOS integrated circuit. The circuits for wireless power transfer can also be contained or otherwise provided on the flexible printed-circuit board.

The exemplary system can further comprise an externally mounted relay station configured to wirelessly communicate with the wireless neural interface device. The exemplary system can also comprise an implanted relay station to be placed or otherwise provided in the skull, spine, or soft tissue under the skin configured to wirelessly communicate with the wireless neural interface device. The exemplary system can also comprise an external computer connected to the externally mounted relay station.

The plurality of electrodes can be or include an ultrathin polymer-based microelectrode array. The ultrathin polymer-based microelectrode array can be formed using thin-film microfabrication techniques. The plurality of electrodes can be or include a high resolution electrocorticography array for recording at a neural surface. The plurality of electrodes can be or include penetrating electrodes for stimulating deep brain structures.

The exemplary CMOS integrated circuit can be thinned down using thinning techniques including backside grinding and reactive ion etching (RIE). The exemplary CMOS integrated circuit can be coupled to the flexible printed circuit board containing the plurality of electrodes through bonding. The bonding can be performed by, e.g., forming solder bumps on bondpads of the CMOS integrated circuit, aligning to bondpads of the flexible printed circuit board, carrying out standard solder reflow techniques, and applying underfilling epoxy. The bonding can also be performed by, e.g., using anisotropic conductive film (ACF) or anisotropic conductive adhesive (ACA).

According to further exemplary embodiments of the present disclosure, a method of manufacturing a wireless neural interface device for electrical stimulation and recording can be provided. The exemplary method can comprise: providing a complementary metal-oxide-semiconductor (CMOS) integrated circuit that is thinned down such that it is mechanically flexible; fabricating a flexible printed circuit board containing a plurality of electrodes; and coupling the flexible printed circuit board to the CMOS integrated circuit.

For example, the action of providing a complementary metal-oxide-semiconductor (CMOS) integrated circuit that is thinned down such that it is mechanically flexible, can comprise thinning down the CMOS integrated circuit using thinning techniques including backside grinding and/or reactive ion etching (RIE). The action of fabricating a flexible printed circuit board containing a plurality of electrodes, can comprise forming the plurality of electrodes using thin-film microfabrication techniques.

The action of coupling the flexible printed circuit board to the CMOS integrated circuit, can comprise: forming solder bumps on bondpads of the CMOS integrated circuit; aligning the bondpads of the CMOS integrated circuit to bondpads of the flexible printed circuit board; carrying out standard solder reflow techniques; and applying underfilling epoxy. In other embodiments, the action of coupling the flexible printed circuit board to the CMOS integrated circuit, can comprise bonding the flexible printed circuit board to the CMOS integrated circuit using an anisotropic conductive film (ACF) or an anisotropic conductive adhesive (ACA).

In some exemplary embodiments of the present disclosure, the exemplary method can further comprise encapsulating the CMOS integrated circuit using a chemical vapor deposition (CVD) process to deposit a conformal biocompatible exterior.

As described herein, a fully-integrated flexible wireless neural interface platform is manufactured using custom-designed wireless neural signal processing CMOS ICs incorporated into microfabricated polymer-based electrode arrays. The exemplary embodiments can include processes for aggressively thinning a CMOS die to make it better suited for implantable application and for bonding this ultrathin die to a flexible electrode array. Ultrathin die can utilize very particular and delicate handling but offer tremendous upside in interfacing with the biological world. Integrating these wireless die with flexible electrode arrays fabricated with thin-film techniques can offer significantly improved chronic efficacy while prioritizing patient safety by eliminating dangerous percutaneous feedthroughs. Additionally, by enabling a system for independent design of the CMOS IC and the flexible electrode array, the exemplary technology described herein can be applied to various potential applications, from recording from the cortical surface to stimulating deep brain structures, or applied to the peripheral nervous system, all with the basic process largely unchanged. By building further capabilities in the CMOS circuitry and electrode materials, the exemplary technology described herein can also be leveraged for electrochemical sensing and targeted drug delivery to enhance understanding of the nervous system and enable treatment of a new generation of clinical diseases.

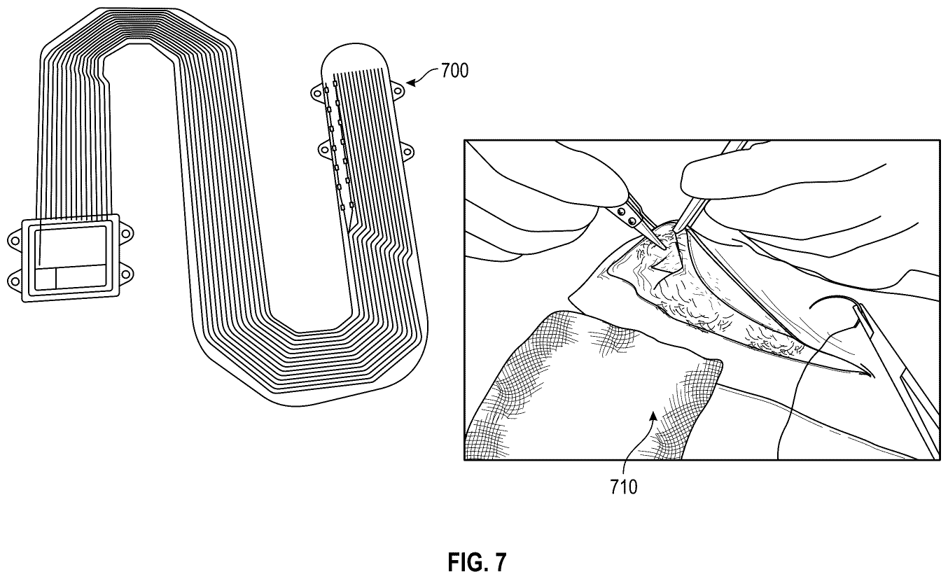

In another exemplary embodiment, an exemplary device 700 can be implanted in a porcine model 710 for stimulation and recording of the spinal cord, as shown in FIG. 7. In this exemplary case, the CMOS chip of the exemplary device 700 can be position subdermally, while the electrode array can be positioned epidurally on the spinal cord.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXEMPLARY REFERENCES

The following references are hereby incorporated by references, in their entireties:
Kensall, J. J., & Wise, D. (1992). An Implantable CMOS Circuit Interface for Multiplexed Microelectrode Recording Arrays. IEEE Journal of Solid-State Circuits, 27(3), 433-443. https://doi.org/10.1109/4.121568.
Jun., J. J., Steinmetz, N. A., Siegle, J. H., Denman, D. J., Bauza, M., Barbarits, B., Lee, A. K., Anastassiou, C. A., Andrei, A., Aydin, C., Barbic, M., Blanche, T. J., Bonin, V., Couto, J., Dutta, B., Gratiy, S. L., Gutnisky, D. A., Häusser, M., Karsh, B., . . . Harris, T. D. (2017). Fully integrated silicon probes for high-density recording of neural activity. Nature, 551(7679), 232-236. https://doi.org/10.1038/nature24636.
Dragas, J., Viswam, V., Shadmani, A., Chen, Y., Bounik, R., Stettler, A., Radivojevic, M., Geissler, S., Obien, M. E. J., Muller, J., & Hierlemann, A. (2017). In Vitro Multi-Functional Microelectrode Array Featuring 59 760 Electrodes, 2048 Electrophysiology Channels, Stimulation, Impedance Measurement, and Neurotransmitter Detection Channels. IEEE Journal of Solid-State Circuits, 52(6), 1576-1590. https://doi.org/10.1109/JSSC.2017.2686580.
Kozai, T. D. Y., Catt, K., Li, X., Gugel, Z. V., Olafsson, V. T., Vazquez, A. L., & Cui, X. T. (2015). Mechanical failure modes of chronically implanted planar silicon-based neural probes for laminar recording. Biomaterials, 37, 25-39. https://doi.org/10.1016/j.biomaterials.2014.10.040.
Yin, M., Borton, D. A., Aceros, J., Patterson, W. R., & Nurmikko, A. V. (2013). A 100-channel hermetically sealed implantable device for chronic wireless neurosensing applications. IEEE Transactions on Biomedical Circuits and Systems, 7(2), 115-128. https://doi.org/10.1109/TBCAS.2013.2255874.
Marblestone, A. H., Zamft, B. M., Maguire, Y. G., Shapiro, M. G., Cybulski, T. R., Glaser, J. I., Amodei, D., Stranges, P. B., Kalhor, R., Dalrymple, D. a, Seo, D., Alon, E., Maharbiz, M. M., Carmena, J. M., Rabaey, J. M., Boyden, E. S., Church, G. M., & Kording, K. P. (2013). Physical principles for scalable neural recording. Frontiers in Computational Neuroscience, 7, 137. https://doi.org/10.3389/fncom.2013.00137.
Nguyen, J. K., Park, D. J., Skousen, J. L., Hess-Dunning, A. E., Tyler, D. J., Rowan, S. J., Weder, C., & Capadona, J. R. (2014). Mechanically-compliant intracortical implants reduce the neuroinflammatory response. Journal of Neural Engineering, 11(5), 056014. https://doi.org/10.1088/1741-2560/11/5/056014.
Weltman, A., Yoo, J., & Meng, E. (2016). Flexible, penetrating brain probes enabled by advances in polymer microfabrication. Micromachines, 7(10). https://doi.org/10.3390/mi7100180.

The invention claimed is:

1. A system for facilitating at least one of electrophysiological recording or stimulation, comprising:
a wireless neural interface device that includes a complementary metal-oxide-semiconductor (CMOS) integrated circuit; and
a flexible printed circuit board that includes a plurality of electrodes coupled to the CMOS integrated circuit.

2. The system of claim 1, wherein a CMOS portion of the wireless neural interface device is located in a subarachnoid space.

3. The system of claim 2, wherein the CMOS portion is configured to be positioned subdermally.

4. The system of claim 1, wherein the wireless neural interface device comprises a radio transceiver for data transmission.

5. The system of claim 4, wherein the radio transceiver is provided on at least one of the CMOS integrated circuit or the flexible printed-circuit board.

6. (canceled)

7. The system of claim 1, wherein the wireless neural interface device comprises one or more circuits which provide a wireless power transfer based on a near-field inductive coupling.

8. The system of claim 7, wherein the one or more circuits are provided on at least one of the CMOS integrated circuit or the flexible printed-circuit board.

9. (canceled)

10. The system of claim 1, further comprising an externally mounted relay station configured to wirelessly communicate with the wireless neural interface device.

11. The system of claim 10, further comprising a computer connected to the externally mounted relay station.

12. The system of claim 1, wherein the plurality of electrodes are an ultrathin polymer-based microelectrode array.

13. The system of claim 12, wherein the ultrathin polymer-based microelectrode array is formed using thin-film microfabrication techniques.

14. The system of claim 1, wherein the CMOS integrated circuit is thinned down using one or more thinning techniques including backside grinding and reactive ion etching (RIE).

15. The system of claim 1, wherein the plurality of electrodes includes at least one of a high resolution electrocorticography array for recording at a neural surface or penetrating electrodes for stimulating one or more deep brain or spinal cord structures.

16. (canceled)

17. The system of claim 1, wherein the CMOS integrated circuit is coupled to the flexible printed circuit board by being bonded to one another.

18. The system of claim 17, wherein the CMOS integrated circuit is bonded to the flexible printed circuit board by forming solder bumps on bondpads of the CMOS integrated circuit, aligning to bondpads of the flexible printed circuit board, performing a solder reflow procedure, and applying an underfilling epoxy.

19. The system of claim 17, wherein the CMOS integrated circuit is bonded to the flexible printed circuit board using an anisotropic conductive film (ACF) or an anisotropic conductive adhesive (ACA).

20. A method for manufacturing a wireless neural interface device for at least one of electrophysiological recording or an electrical stimulation, comprising:
providing a complementary metal-oxide semiconductor (CMOS) integrated circuit that is mechanically flexible by being thinned;
providing a flexible printed circuit board containing a plurality of electrodes; and
coupling the flexible printed circuit board to the CMOS integrated circuit.

21. The method of claim 20, wherein a CMOS portion of a wireless neural interface device that includes the CMOS integrated circuit is located in a subarachnoid space.

22. The system of claim 21, wherein the CMOS portion is configured to be positioned subdermally.

23. The method of claim 20, wherein the CMOS integrated circuit is provided by thinning the CMOS integrated circuit using at least one a backside grinding procedure or a reactive ion etching (RIE) procedure.

24. The method of claim 20, wherein the flexible printed circuit board is provided by forming the plurality of electrodes using at least one thin-film microfabrication procedure.

25. The method of claim 20, wherein the flexible printed circuit board is coupled to the CMOS integrated circuit by:
forming solder bumps on bondpads of the CMOS integrated circuit;
aligning the bondpads of the CMOS integrated circuit to bondpads of the flexible printed circuit board;
performing at least one solder reflow procedure; and
applying underfilling epoxy to the CMOS integrated circuit.

26. The method of claim 25, further comprising encapsulating the CMOS integrated circuit using a chemical vapor deposition (CVD) process to deposit a conformal biocompatible exterior.

27. The method of claim 20, wherein the flexible printed circuit board is coupled to the CMOS integrated circuit by bonding the flexible printed circuit board to the CMOS integrated circuit using at least one of an anisotropic conductive film (ACF) or an anisotropic conductive adhesive (ACA).

28. The method of claim 27, further comprising encapsulating the CMOS integrated circuit using a chemical vapor deposition (CVD) process to deposit a conformal biocompatible exterior.
